(19) United States
(12) Patent Application Publication
(10) Pub. No.: US 2003/0077302 A1
(43) Pub. Date: Apr. 24, 2003

(54) Diclofenamide suspension gel

(76) Inventors: Gudrun Claus-Herz, Berlin (DE); Gunther Bellmann, Berlin (DE)

Correspondence Address: Law Department, Bausch & Lomb Incorporated, One Bausch & Lomb Place, Rochester, NY, 14604-2701, US

(21) Appl. No.: 10/181,797

(22) PCT Filed: Jan. 18, 2001
(86) PCT No.: PCT/EP01/00557

(30) Foreign Application Priority Data
Jan. 20, 2000 (DE) 100022332

(51) Int. Cl. A61K 31/195; A61K 9/00

(52) U.S. Cl. 424/400; 514/567

(57) ABSTRACT

The present invention relates to ophthalmic compositions in the form of suspension gels of the free acid of the diclofenamide for treating glaucoma. The invention also relates to the use of suspension gels containing the free acid of the diclofenamide for treating primary and/or secondary open-angle glaucomas, for reducing the intra-ocular pressure and for treating ocular hypertension.

This invention concerns a new ophthalmic composition in the form of a suspension gel that contains diclofenamide in the form of free acid. In addition, this invention concerns the use of diclofenamide suspension gel to treat ocular hypertension and for therapy of primary and secondary open-angle glaucomas.

The use of diclofenamide (INN: dichlorphenamide, 4,5-dichlorbenzene-1,3-disulfonamide) to reduce intraocular pressure has long been known. The free acid of diclofenamide is in the prior art almost exclusively systemically. Diclofenamide acts as a carboanhydrase inhibitor, which blocks the multifunctional enzyme carboanhydrase. In the eye carboanhydrase (II) is reversibly inhibited, which in the end leads to a reduction of the aqueous secretion and consequently to a reduction of intraocular pressure.

Carboanhydrase inhibitors like diclofenamide are used in the treatment of glaucoma, a generative disease of the eye, which is accompanied by an increase of the intraocular pressure. Besides the systemic administration of diclofenamide preparations for topical use have been known since the 1980s and are increasingly being used. While the free acid of diclofenamide is mostly used for systemic administration, up to now only the alkali metal salts of diclofenamide have been used for topical application.

In particular, salt-forming carboanhydrase inhibitors have been used in ophthalmic compositions for topical application in the treatment of ocular hypertension.

For example, the European Patent Application EP-A-033 042 describes the use of various carboanhydrase inhibitors in the form of their alkali metal salts to make ophthalmic preparations. Besides examples of ophthalmic solutions of methazolamide sodium salts, in some cases thickened to be gel-like with hydroxypropylmethylcellulose, the use of diclofenamide dipotassium salt is also described in EP-A-033 042, among other places.

The use of monoalkali metal salts of dibasic carboanhydrase inhibitors in ophthalmic preparations for topical use is described in European Patent Application EP-A-036 351. The corresponding ophthalmic compositions are solutions of the active agent or solution gels.

Lotti et al. (Graefe's Archive for Clinical and Experimental Ophthalmology 1984, 222:13-19) conducted a study on the topical ocular hypotensive activity and ocular penetration of diclofenamide sodium in rabbits. It was found that an aqueous solution of the sodium salt of diclofenamide is capable of producing a clear and long lasting reduction of the intraocular pressure. Lotti et al. reported that the use of solutions of diclofenamide sodium salt leads to a clearly stronger and longer reduction of the intraocular pressure than suspensions of the free acid of diclofenamide.

The aqueous humor values in the study by Lotti et al. were clearly at a lower level when using suspensions of diclofenamide than with solutions of sodium diclofenamide. However, it was also established by Lotti et al. that, although single applications of sodium diclofenamide solutions are well tolerated, the repeated use of diclofenamide sodium solutions triggers severe undesirable side effects in rabbits.

In order to overcome these known disadvantages of the ophthalmic compositions of the prior art that contain diclofenamide, this invention is based on the task of making available an ophthalmic composition containing diclofenamide that is capable of effectively reducing the intraocular pressure and at the same that is well tolerated.

Another task of the invention is to make available a suspension gel of the free acid of diclofenamide that can be used for systemic administration in place of diclofenamide preparations.

This invention solves the above tasks by making available an ophthalmic composition for glaucoma treatment in the form of a suspension gel that contains the free acid of diclofenamide, preservatives, gel forming agents, agents to adjust the pH value, water, and optionally other conventional additives.

Surprisingly, in contrast to the study by Lotti et al., it was found that the formulation of diclofenamide as a free acid in suspension gels leads to ophthalmic preparations that can achieve comparable effects to those that can be achieved through the systemic administration of diclofenamide.

The formulation of [diclofenamide] the containing ophthalmic composition with diclofenamide in the form of the free acid as suspension gels in accordance with this invention leads to highly effective and well tolerated drugs for treatment of primary and secondary open-angle glaucomas and ocular hypertension.

To make the suspension gels in accordance with the invention, the free acid of diclofenamide has to be micronized. This micronization takes place by the methods that are conventional in the prior art, for example by grinding in various kinds of mills, trituration and screening.

The micronized particles for use in ophthalmic compositions in accordance with the invention preferably have particle sizes less than 90 μm, preferably less than 50 μm, especially preferably less than 25 μm.

Chiefly more than 50%, preferably more than 60%, preferably more than 80% and most preferably more than 99% of the diclofenamide particles have a particle size less than 90 μm.

In accordance with the invention more than 50%, preferably more than 60%, preferably more than 80% and even more preferably more than 95% of the diclofenamide particles suitably have a particle size less than 50 μm.

It is advantageous if more than 10%, preferably more than 20%, more preferably more than 30% and still more preferably more than 50%, furthermore preferably 80% and most preferably more than 95% of the diclofenamide particles have a particle size less than 25 μm.

It is especially preferred if 100% of the diclofenamide particles are smaller than 90 μm, more preferably smaller than 50 μm and most preferably smaller than 25 μm.

Before further processing to a suspension gel the micronized diclofenamide is preferably sterilized.

All of the polymer compounds suitable for producing stable suspension gels can be used as gel forming agents. In particular, the gel forming agents are chosen from the group consisting of polyacrylic acid, cellulose ethers, polyvinyl alcohol and polysaccharides. These can, as desired, be of synthetic or natural origin.

The use of hydrogels bases on polyacrylic acid and/or cellulose ethers is especially preferred. Polyacrylic acid gels are in particular preferred, especially ones based on carbomers of the Carbopol® type, which are commercially available from B. F. Goodrich. Especially preferred is the use of Carbopol 980 as gel forming agent in suspension gels in accordance with the invention.

Preferred embodiments of the diclofenamide suspension gels in accordance with the invention contain polyacrylic acid gels like Carbopol, especially Carbopol 980, in an amount from >0 to 10 wt %, preferably 0.01 wt % to 5 wt %, more preferably 0.05 wt % to 1 wt %, even more preferably 0.1 wt % to 0.5 wt % and most preferably 0.2 wt % to 0.4 wt %.

In order to avoid possible contamination of the gel during application, the ophthalmic compositions in accordance with the invention are preserved through the addition of a preservative agent. All of the substances that are well known to the specialist and suitable for application to the eye can be used as preservatives, for example benzyl alcohol, benzalconium chloride or other quaternary ammonium compounds, chlorhexidine diacetate or dicluconate [sic; digluconate], thiomersal etc. Especially preferred is the use of benzododecinium salts, especially benzododecinium chloride.

Preferably sorbitol is used as another additive in the composition in accordance with the invention. In preferred embodiments of the diclofenamide suspension gels in accordance with the invention sorbitol and/or a polyacrylic acid gel are contained in an amount from 0 to 10 wt %, preferably 2 to 8 wt %, especially preferably 3 to 6 wt % and most preferably between 4 wt % and 5 wt %.

Glycerol, mannitol and/or dextrose can also be used instead of sorbitol.

Mineral acids and bases are used to adjust the pH value of the suspension gels in accordance with the invention. The use of sodium hydroxide and/or hydrochloric acid is especially preferred.

The pH value of the ophthalmological composition in accordance with the invention lies between pH 6.8 and pH 7.6, preferably pH 7-7.5, more preferably pH 7.1-7.5 and most preferably between pH 7.25 and pH 7.35.

Preferred embodiments of the suspension gels in accordance with the invention have a viscosity between 1,000 and 8,000 mPa.sec, preferably between 2,000 and 6,000 mPa.sec and especially preferably between 2,500 and 5,500 mPa.sec and even more preferably between 2,800 and 4,500 mPa.sec.

Preferably the viscosity of the gel composition in accordance with the invention is set so that the contact time in the eye is ≧1 min, especially at least ≧5 min, preferably ≧10 min, more preferably ≧15 min, even more preferably ≧20 min and most preferably ≧30 min. The contact time is the time measured from the application of the gel in accordance with the invention up to the time at which the gel has been completely washed out of the eye together with lactrimal fluid.

The active agent content of the suspension gels in accordance with the invention is 0.1 to 10 wt %, preferably 0.1 to 5 wt %, more preferably 0.5 wt % to 4 wt %, even more preferably 1 wt % to 3 wt % and most preferably 1.5 wt %-2 wt % of the free acid of diclofenamide.

Suspension gels that have an active agent content between 2 wt % and 7 wt % free acid of diclofenamide are more preferred.

An advantage of the suspension gels of the free acid of diclofenamide in accordance with the invention over the solutions and solution gels of diclofenamide potassium or sodium salts that are known in the prior art is in particular their clearly better tolerability. For example, no undesirable eye irritations like burning and the like arise.

The preparation of the suspension gels in accordance with the invention takes place by the usual methods of the prior art. The micronized active agent is sterilized and added to a sterile suspension gel matrix. Then it is homogenized so that a completely uniform distribution of the suspended particles is achieved.

The ophthalmic diclofenamide compositions in accordance with the invention are suitable for pressure-reducing treatment of primary and secondary open-angle glaucomas and for treatment of ocular hypertension.

The tolerability of the gel formulations in accordance with the invention, including the free acid of diclofenamide, over a period of administration of 6 weeks is very good, i.e., no undesired side effects in the eye occur.

Weight data, unless otherwise specified, refer to the overall composition of the gel composition in accordance with the invention.

Suspension gels in the sense of this invention are gels in which the active agent in undissolved form is finely suspended as solid particles in the gel base, or carrier matrix.

The invention is illustrated in more detail below by means of some embodiment examples.

### EXAMPLES 1 TO 6

6 Diclofenamide suspension gels with free diclofenamide acid concentrations from 0.1 to 5 wt % were prepared. The compositions of some examples are given in the following Table 1. All percentage data are understood to be percent by weight with respect to the total composition.

The prepared suspension gels have the physical properties given in Table 2.

TABLE 1

| | Example 1 | Example 2 | Example 3 | Example 4 | Example 5 | Example 6 |
|---|---|---|---|---|---|---|
| | Diclo- | Diclo- | Diclo- | Diclo- | Diclo- | Diclo- |
| Ingredients | fenamide | fenamide | fenamide | fenamide | fenamide | fenamide |
| | Gel 5% | Gel 3% | Gel 2% | Gel 1% | Gel 0.5% | Gel 0.1% |
| Diclofenamide | 5% | 3% | 2% | 1% | 0.5% | 0.1% |
| (free acid) | | | | | | |
| Carbopol 980 NF | 0.2% | 0.2% | 0.2% | 0.2% | 0.2% | 0.2% |
| Benzododecinium | 0.01% | 0.01% | 0.01% | 0.01% | 0.01% | 0.01% |
| chloride | | | | | | |
| Sorbitol | 4.802% | 4.898% | 4.9% | 4.8% | 4.89% | 4.795% |
| Sodium hydroxide | 0.079% | 0.079% | 0.078% | 0.077% | 0.079% | 0.077% |
| Water for injection | 100% | 100% | 100% | 100% | 100% | 100% |

TABLE 2

| | Example 1 | Example 2 | Example 3 | Example 4 | Example 5 | Example 6 |
|---|---|---|---|---|---|---|
| | Diclo- | Diclo- | Diclo- | Diclo- | Diclo- | Diclo- |
| Properties | fenamide | fenamide | fenamide | fenamide | fenamide | fenamide |
| | Gel 5% | Gel 3% | Gel 2% | Gel 1% | Gel 0.5% | Gel 0.1% |
| pH | 7.11 | 7.02 | 7.43 | 7.22 | 7.12 | 7.09 |
| Osmol- | 322 mosmol/kg | 316 mosmol/kg | 301 mosmol/kg | 300 mosmol/kg | 303 mosmol/kg | 296 mosmol/kg |
| ality | | | | | | |
| Viscosity | 3857 mPa s | 4170 mPa s | 2848 mPa s | 4060 mPa s | 3451 mPa s | 3539 mPa s |

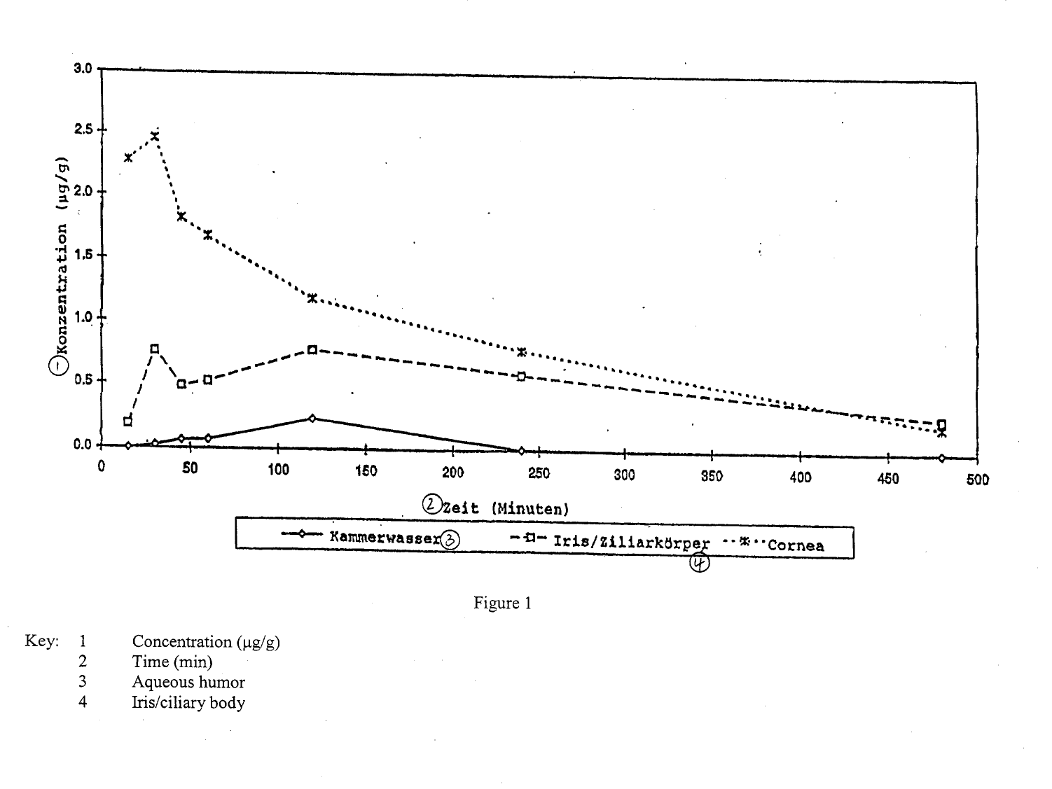

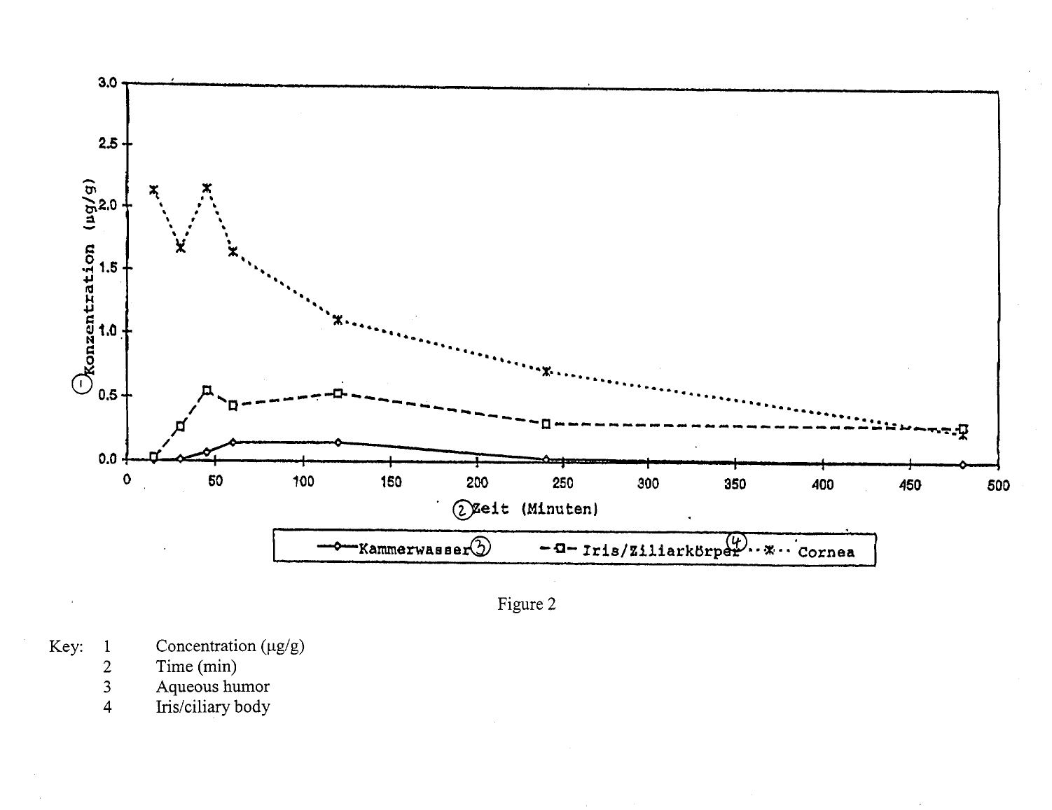

An intraocular bioavailability study of a single dose in rabbits was carried out with the 3 and 5% gels from Examples 1 and 2. For this the concentrations of diclofenamide in the vitreous humor in the iris-ciliary body at the cornea were measured after various intervals of time. The measured concentrations are plotted in FIGS. 1 and 2 against time.

Here FIG. 1 shows the concentration time curves of diclofenamide when using the suspension gel from Example 1, and

FIG. 2 shows the concentration time curves of diclofenamide when using the suspension gel from Example 2.

It can be seen from FIGS. 1 and 2 that the capacity of the free acid of diclofenamide to penetrate from the suspension gel through the cornea is relatively high. The iris-ciliary body level of the topically applied diclofenamide from the suspension gel in accordance with the invention is at a comparable level to systemic administration over 8 hours.

The tolerability of the tested gel formulation over a test period of 6 weeks is very good; with the tested suspension gels applied 3 times daily over a period of 6 weeks no eye irritations or tissue damage to the eye could be detected. Likewise, no signs of systemic side effects were found.

1. An ophthalmic composition for glaucoma treatment in the form of a suspension gel containing the free acid of diclofenamide, preservatives, gel forming agents, agents to adjust the pH value, water and optionally other conventional additives.

2. An ophthalmic composition as in claim 1, which is characterized by the fact that the gel forming agent is chosen from the group consisting of polyacrylic acid, cellulose ethers, polyvinyl alcohol and polysaccharides.

3. An ophthalmic composition as in one of claims 1 or 2, which is characterized by the fact that benzododecinium chloride is used as preservative.

4. An ophthalmic composition as in one of claims 1 to 3, which is characterized by the fact that Carbopol, preferably Carbopol 980 is used as gel forming agent.

5. An ophthalmic composition as in one of claims 1 to 4, which is characterized by the fact that the suspension gel contains between 0.1 and 10 wt % and preferably 0.1 and 5 wt % of the free acid of diclofenamide.

6. An ophthalmic composition as in one of claims 1 to 5, which is characterized by the fact that the suspension gel contains 0 to 10 wt %, preferably 2 to 8 wt % and especially preferably 3 to 6 wt % sorbitol.

7. An ophthalmic composition as in one of claims 1 to 6, which is characterized by the fact that the viscosity of the suspension gel is between 1,000 and 8,000 mPa.sec, preferably between 2,000 and 6,000 mPa.sec and especially preferably between 2,500 and 5,500 mPa.sec.

8. An ophthalmic composition as in one of claims 1 to 7, which is characterized by the fact that the diclofenamide is micronized to particle sizes smaller than 90 μm, preferably smaller than 50 μm, especially preferably smaller than 25 μm.

9. The use of an ophthalmic composition as in one of claims 1 to 8 to reduce intraocular pressure.

10. The use of an ophthalmic composition as in one of claims 1 to 8 to treat primary and/or secondary open-angle glaucomas.

11. The use of an ophthalmic composition as in one of claims 1 to 8 to treat ocular hypertension.
